(19) United States
(12) Patent Application Publication
(10) Pub. No.: US 2018/0325973 A1
(43) Pub. Date: Nov. 15, 2018

(54) SKIN CARE COMPOSITION

(71) Applicant: ISN PHARMA SP.Z.O.O., Warszawa, Woj Mazowieckie (PL)

(72) Inventor: Sylwia NOWAK, Woj. Lódzkie (PL)

(21) Appl. No.: 15/774,670

(22) PCT Filed: Nov. 11, 2016
(86) PCT No.: PCT/IB2016/056808

§ 371 Date: May 9, 2018

(30) Foreign Application Priority Data
Nov. 12, 2015 (PL) 414758
Aug. 3, 2016 (GB) 1613401.7

(51) Int. Cl. A61K 36/41; A61K 9/00; A61K 47/14; A61K 47/44; A61K 47/10; A61K 47/22; A61K 9/70; A61K 9/06; A61K 8/9789; A61Q 19/00; A61P 17/06; A61P 37/00

(52) U.S. Cl. CPC A61K 36/41; A61K 9/0014; A61K 47/14; A61K 47/44; A61K 47/10; A61P 37/00; A61K 9/7023; A61K 9/06; A61K 8/9789; A61Q 19/00; A61P 17/06; A61K 47/22

(57) ABSTRACT

The present invention relates to skin care compositions comprising extract or juice from the plant Kalanchoe daigremontiana for topical administration to human skin and methods of making said compositions. The compositions are therapeutically effective for hydrating and conditioning the skin and are shown to be clinically effective at treating dermal conditions or skin disorders, in particular, psoriasis, atopic dermatitis or allergic contact dermatitis.

This invention relates to a skin care composition comprising extract, for example the juice, from Kalanchoe daigremontiana for topical administration to human skin. In particular, the invention relates to a method of making such a composition and to cosmetic and medical uses thereof.

### BACKGROUND

In the field of skin care, a wide variety of products have been developed and commercialised for enhancing skin condition.

Such products are typically formulated as gels, creams, rinses, film-forming compositions, transdermal patches, sprays, or pastes aimed at providing easy application to the skin and effective absorption of components in the formulation. Such formulations must also be stable for reasonable storage periods.

However, the art is continually seeking improved solutions wherein a skin care product both enhances skin condition and is particularly effective in the treatment of one or more common medical complaints associated with the skin. For example, compositions formulated to combat common adverse dermal disorders such as dermatitis, acne, rosacea, psoriasis, atopic dermatitis and/or allergic contact dermatitis. Skin products directed at alleviating dermal disorders may therefore also frequently include a therapeutically active agent, such as a naturally derived plant extracts, such as aloe vera, or a manufactured ingredient or chemical, such as benzoyl peroxide or hexachlorophene.

In the case of some particular dermal complaints, such as psoriasis and atopic dermatitis, the routinely used treatment prescribed by dermatologists includes application of a formulation comprising a glycocorticosteroid. Steroids are prescription drugs and require frequent application of 1 to 3 times per day for at least 2 weeks. However, steroids are undesirable for a number of reasons: they are expensive for the patient and/or health services and often lead to serious side effects which include inter alia: systemic hormonal changes, skin atrophy, eczema, steroid acne, erythema, vascular purpura, telangiectasia, skin irritation, e.g. burning sensation, reddening, extreme dryness and sometimes bacterial or mycotic superinfections. Moreover, the curative effects of steroids are short-term and after the end of the treatment period the lesions and other physical symptoms associated with psoriasis and atopic dermatitis can re-appear in an aggravated form.

Other treatment options for dermal conditions such as psoriasis include compositions comprising urea, salicylic acid and/or lactic acid. However, these treatments fail to provide a sufficient long term solution for patients; they reduce the superficial appearance of squama on the surface of psoriatic plaque but do not prevent or alleviate regenerating lesions or allow the skin to heal fully. Further, by using such preparations, skin cannot be exposed to sunlight.

Although, medicinal plants are commonly applied in the field of topical formulations, there is a continuing need to identify extracts and develop useful formulations of those extracts to yield solutions for both skin enhancement and clinical treatment of common dermatological conditions. This is particularly true of skin psoriasis, which affects from 1 to 3% of the population.

Polish patent application no. PL 398082 discloses a composition which contains from 36% to 52% w/w of phyto product in the form of juice or leaf pulp of two plants: Kalanchoe daigremontiana and Kalanchoe pinnata. It is disclosed that the composition improves animal health and condition. For example: poultry resistance to pathogens is greater and the susceptibility to pathogens (micro-organisms) is reduced. However, there is no indication that such extracts from either of these plants would be useful in the health of humans or be useful in the treatment of a particular human condition.

The applicant has recognised a continuing need to identify and develop a product which enhances skin condition and provides a clinically effective treatment for common dermal complaints.

### SUMMARY OF INVENTION

The present invention relates to skin care compositions comprising the extract from Kalanchoe daigremontiana for administration to human skin. The compositions are therapeutically effective for both skin conditioning and combating skin disorders.

The extract may include or comprise the juice of from Kalanchoe daigremontiana.

The invention particularly concerns a composition for topical administration comprising a therapeutically effective amount of Kalanchoe daigremontiana extract. The extract may be derived from the juice of the pulp of the Kalanchoe daigremontiana plant and/or from the juice derived from the leaves and/or the stalk.

In one embodiment, the composition is formulated with at least one cosmetically or pharmaceutically acceptable excipient.

The inventors have shown that such a composition is surprisingly useful in the regeneration and recovery of the natural protective layer of the skin. It is also shown that the composition is surprisingly effective in soothing irritation and inflammation of human skin.

Such a composition is advantageously also found to reduce or eliminate roughness, exfoliation, pulling and burning and soothes itching after a single application. The extract from Kalanchoe daigremontiana appears to be a highly effective active ingredient for re-conditioning and regenerating healthy skin cells. The composition of the invention containing Kalanchoe daigremontiana, therefore presents a new and surprising application in the area of topical plant-derived therapeutic compositions.

Preferably, the composition comprises 1-30% w/w of Kalanchoe daigremontiana extract or juice.

More preferably the composition comprises 5-15% w/w of Kalanchoe daigremontiana extract or juice, most preferably approximately 9-10% w/w.

In preferred embodiments, the composition may be an epicutaneous formulation and/or formulated as a powder, paste, cream, foam, gel, lotion, ointment or shampoo, for ease of application to a preferred part of the body e.g. the scalp.

In a preferred embodiment, the composition is formulated as a cream. Advantageously, such a cream formulation has excellent skin-absorption properties and enhances the therapeutic effect of the plant extract.

In one embodiment, the composition additionally comprises demineralized water, preferably, 35-75% w/w demineralized water and most preferably approximately 35% w/w.

The at least one excipient may comprises glycerol monostearate, preferably 4-20% w/w glycerol monostearate and most preferably approximately 20% w/w.

Moreover, the excipient or excipients in a preferred embodiment of any of the above-mentioned compositions of the invention includes a mixture of fats comprising glycerol monostearate and one or more of beeswax, coconut oil, cetyl alcohol, shea butter and Cannabis sativa (Hemp) Seed Oil.

In one embodiment, the composition comprises 6-65% w/w of the mixture of fats.

Preferably, the composition also comprises 4-28% w/w of beeswax and/or 4-40% w/w of coconut oil and/or 2-25% w/w of cetyl alcohol and/or 2-10% w/w of shea butter.

In one embodiment the composition additionally comprises allantoin, preferably 0.25-0.5% w/w allantoin, which may enhance the therapeutic effect of the formulation.

In a further embodiment, the composition further comprises a preservative, such as phenoxyethanol, preferably 0.5 to 1% w/w phenoxyethanol and most preferably 1% w/w. The preservative prolongs the microbiological stability of the finished product allowing the composition to be stored and used for a longer period.

Further, it is noted that such embodiments have minimal risk of allergic reactions as the applicants have formulated this naturally derived-plant product composition in the absence of fragrance or artificial colourings.

Further, the invention maybe formulated in accordance any of the above preparations for use with or within a transdermal patch. The invention therefore extends to a dermal patch comprising one or more of any of the above described compositions.

The invention also concerns a composition for topical administration comprising a therapeutically effective amount of Kalanchoe daigremontiana extract, in accordance with any of the before-described compositions, for improving skin condition. Usefully, the composition has been found by patients to enhance the softness, smoothness and hydration of the skin and to promote suppleness after only a single application. Furthermore, the composition of the invention has no adverse side effects.

The invention further concerns a composition comprising a therapeutically effective amount of Kalanchoe daigremontiana extract, in accordance with any of the before described compositions, for use in the treatment of a dermal condition, preferably a human dermal condition.

In a preferred embodiment the dermal condition is skin/xerosis, psoriasis, rosacea, ichthyosis, keratosis, keratoderma, dermatitis, pruritus or eczema. In one embodiment, where the skin relates to the scalp particularly, the dermal condition maybe seborrheic dermatitis or scalp psoriasis.

In the pharmaceutical and cosmetics market there are no highly effective, natural skin care preparations useful in treating dermal conditions that share overlapping or common symptoms. However, surprisingly, the applicants have found that regular application of the composition of the invention substantially reduces or eliminates dermatological ailments and symptoms common to several of the dermal conditions leading to the improvement of the well-being and life quality of the patients.

In a further embodiment, the dermal condition is psoriasis, atopic dermatitis and/or allergic contact dermatitis. The composition is shown to have an excellent therapeutic effect in the treatment of such disorders: effecting a substantial or complete reduction in lesions and co-related physical symptoms associated with such disorders. It is also shown to provide a regenerating effect in the skin of the patients with these conditions. Furthermore, the composition of the invention does not cause or result in serious side effects that are typically associated with therapeutically active compositions currently prescribed for treating such conditions.

In a further embodiment, the invention concerns a composition for use in treating one or more of the above defined conditions, wherein the composition is applied once, or more preferably, twice daily. For intensive treatment the treatment can involve up to 4 applications daily.

In yet a further embodiment, the invention concerns a composition for use in treating one or more of the above-defined conditions, wherein the duration of the treatment is at least a daily application, preferably a twice-daily application, for at least 5 consecutive days, preferably at least 2 weeks, more preferably 6 weeks, and even more preferably 14 weeks.

In a further embodiment, the dermal condition is psoriasis and optionally the treatment duration is 4 to 14 weeks, most preferably 5 to 8 weeks.

In a further embodiment, the dermal condition is contact dermatitis and optionally the treatment duration is 1 to 14 days, most preferably 5 to 10 days.

In a further embodiment, the dermal condition is atopic dermatitis and optionally the treatment duration is 3 days to 98 days, most preferably 7 days to 35 days.

The invention also relates to a method of enhancing skin condition, comprising topically administering to human skin in need thereof, a composition comprising Kalanchoe daigremontiana extract or juice, in accordance with any of the before described compositions.

The invention concerns a method of treating a dermal condition, comprising topically administering to human skin in need thereof, a composition comprising Kalanchoe daigremontiana extract or juice, in accordance with any of the before described compositions.

The invention further concerns a method of treating psoriasis, atopic dermatitis or allergic contact dermatitis, comprising topically administering to human skin in need thereof, a composition comprising Kalanchoe daigremontiana extract or juice, in accordance with any of the before described compositions.

The invention also comprises a process of producing a topical composition comprising: combining demineralized water, optionally with allantoin, with a mixture of fats comprising glycerol monostearate and one or more of beeswax, coconut oil, cetyl alcohol, shea butter and Cannabis sativa (Hemp) Seed Oil together with Kalanchoe daigremontiana extract or juice to obtain a smooth homogenous mixture.

The invention also comprises a method for making a topical composition comprising the steps of: heating a solution of demineralised water, preferably at 30 to 75 degrees C.; heating a mixture of fats comprising one or more of beeswax, coconut oil, cetyl alcohol, shea butter, Cannabis sativa (Hemp) Seed Oil and glycerol monostearate, preferably at 30-75 degrees C., and stirring until smooth; combining the mixture and solution and heating together, preferably at 30-75 degrees C., and stirring until a smooth and homogenous texture is obtained, followed by cooling, preferably to 20-30 degrees C.; and adding a therapeutically effective amount of Kalanchoe daigremontiana extract or juice to the resulting mixture.

In one embodiment, heating the demineralised water is preferably at 30-75 degrees C.

In a further embodiment, heating the mixture of fats is preferably at 30-75 degrees C.

In one embodiment, heating the combined mixture of fats and solution together is at 30-75 degrees C.

In one embodiment, allantoin, preferably 0.25-0.5% w/w, is added to the demineralised water.

In one embodiment, the method comprises a further final step of homogenising the mixture.

Preferably, the composition comprises 1-30% w/w of Kalanchoe daigremontiana extract or juice. More preferably the composition comprises 5-15% w/w of Kalanchoe daigremontiana extract or juice, most preferably approximately 9-10% w/w.

In one embodiment, the composition additionally comprises demineralized water, preferably, 35-75% w/w demineralized water and most preferably approximately 35% w/w.

The at least one excipient may comprises glycerol monostearate, preferably 4-20% w/w glycerol monostearate and most preferably approximately 20% w/w.

In one embodiment, the composition comprises 6-65% w/w of the mixture of fats.

The mixture of fats includes glycerol monostearate and one or more of beeswax, coconut oil, cetyl alcohol, shea butter and Cannabis sativa (Hemp) Seed Oil.

Preferably, the composition comprises 4-28% w/w of beeswax and/or 4-40% w/w of coconut oil and/or 2-25% w/w of cetyl alcohol and/or 2-10% w/w of shea butter.

In a further embodiment, the composition further comprises a preservative, such as phenoxyethanol, preferably 0.5 to 1% w/w phenoxyethanol and most preferably 1% w/w.

Preferably, the composition also comprises allantoin.

In a further aspect, the invention relates to a topical composition obtained by any of the above defined methods.

The extract may include or comprise the juice from any part of Kalanchoe daigremontiana plant.

Other aspects, features and embodiments of the invention will be more fully apparent from the ensuing examples and appended claims.

### BRIEF DESCRIPTION

The following figures provide photographic evidence of the results of a clinical study wherein a topical cream formulation according of the invention was tested. The formulation of the cream is in accordance with a preferred embodiment, such as Example 4.

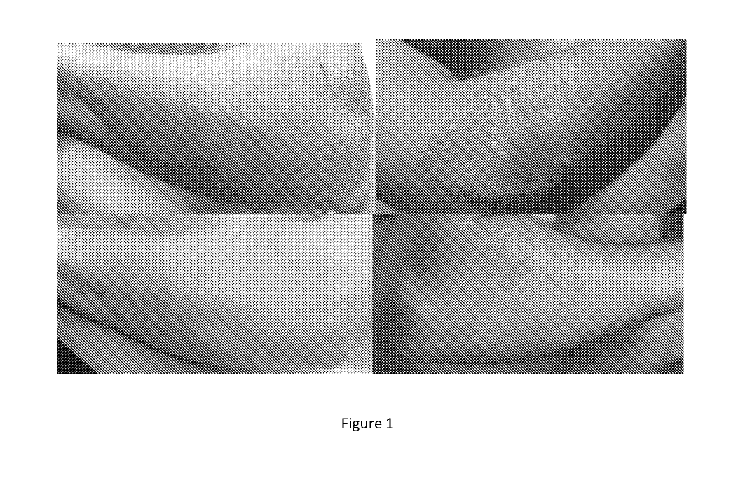

FIG. 1 shows the before and after photos of a male suffering psoriasis, treated with a composition of the invention.

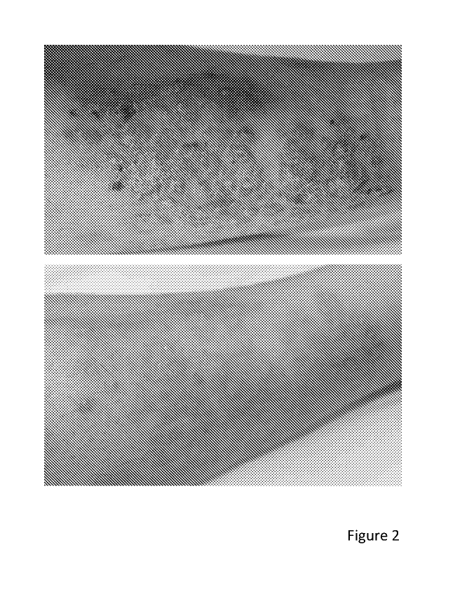

FIG. 2 shows the before and after photos of a female suffering psoriasis, treated with a composition of the invention.

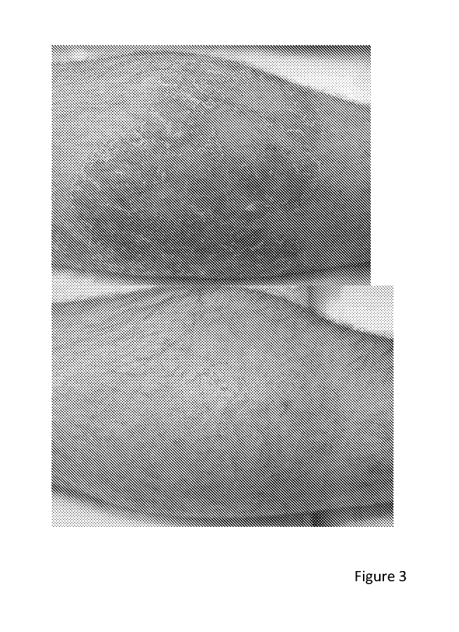

FIG. 3 shows the before and after photos of a male suffering psoriasis, treated with a composition of the invention.

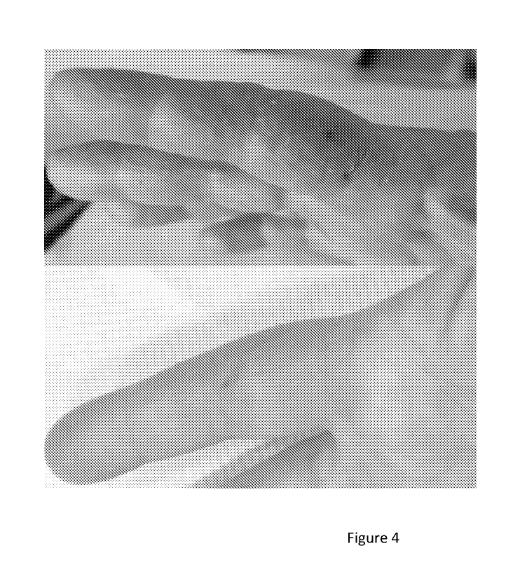

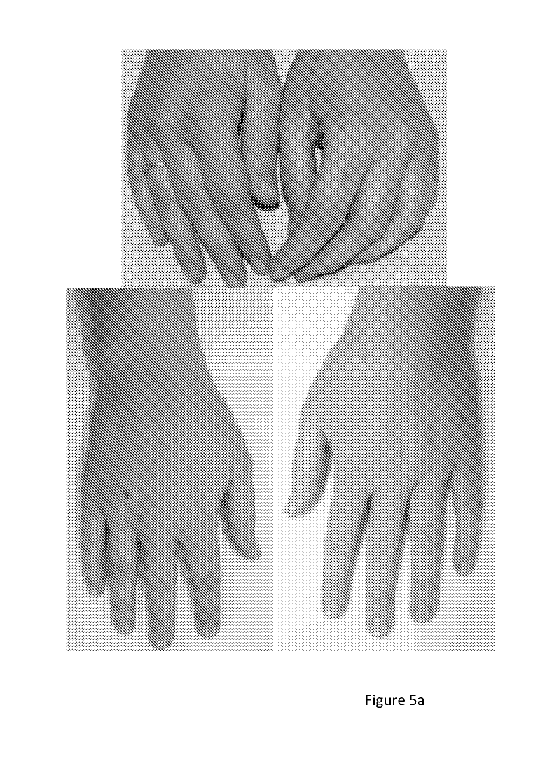

FIG. 4 shows the before and. after photos of a female suffering allergic contact dermatitis, treated with a composition of the invention

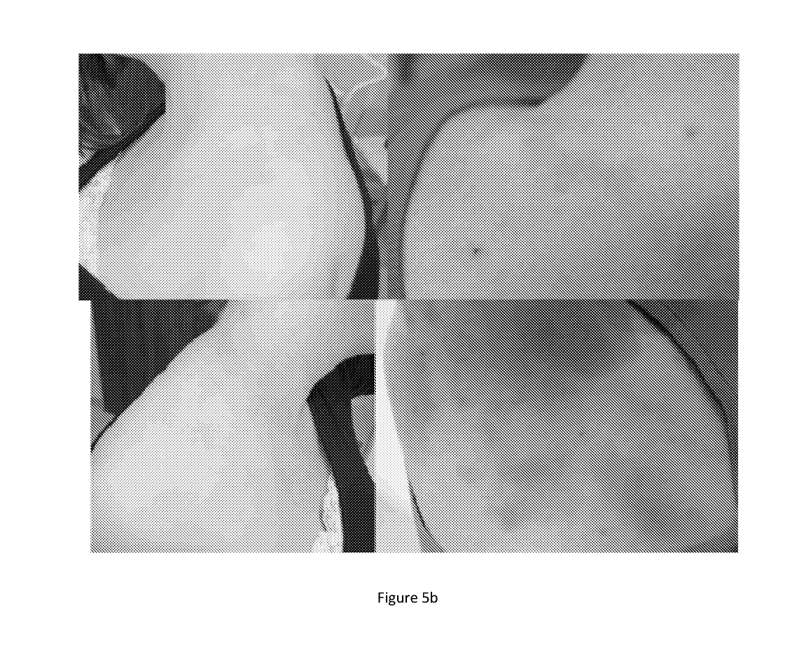

FIG. 5 a/b shows the before and after photos of a female suffering atopic dermatitis, treated with a composition of the invention. Note: FIG. 5b shows the after treatment photos on the left.

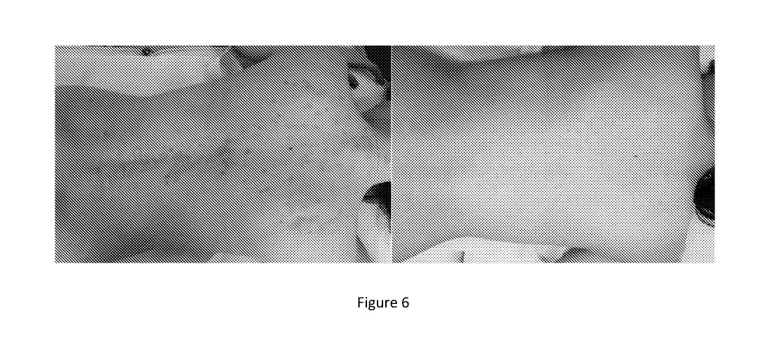

FIG. 6 shows the before and after photos of a female suffering psoriasis, treated with a composition of the invention.

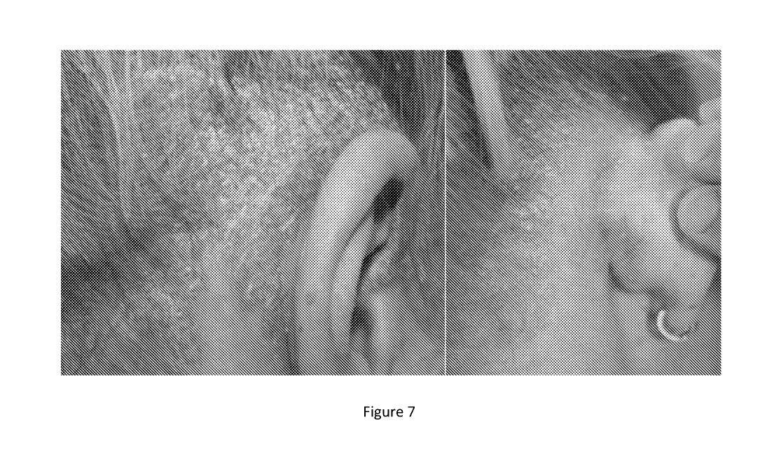

FIG. 7 shows the before and after photos of a female suffering psoriasis, treated with a composition of the invention.

### DETAILED DESCRIPTION

The present invention relates to compositions that are topically administered to improve the character of the skin and to combat adverse skin conditions.

Compositions of the invention are usefully employed as skin moisturizers, skin softening agents, skin debridement agents, etc.

In cosmetic formulations, the compositions of the invention may be used with added ingredients that are solely cosmetic. Alternatively, the cosmetic formulation may include ingredients that are both cosmetically efficacious and therapeutically effective, e.g., so-called “cosmeceutical” ingredients.

Compositions of the invention may also be utilised for treatment clinical dermal conditions and adverse physiological states manifesting dermally, including, without limitation, dry skin/xerosis, psoriasis, ichthyosis, keratosis, keratoderma, dermatitis, pruritus, and eczema.

Conditions found to be particularly well treated by the composition of the invention include psoriasis, atopic dermatitis or allergic contact dermatitis.

As used herein, references to compositional ingredients in percent by weight refers to weight percentages (% w/w) based on the total weight of the composition or formulation.

In various embodiments of the invention, the compositions described herein may comprise, consist or consist essentially of the specified ingredients or specific ones thereof. It will be understood that the formulations of the invention may be widely varied, as regards the absolute amounts and relative proportions thereof, in relation to specific examples, and illustrative compositions.

The invention can be illustrated with the following preferred example compositions:

### Example 1

1-30% w/w of the extract or juice from Kalanchoe daigremontiana

4-20% w/w of the glycerol monostearate

4-28% w/w of the beeswax

35-75% w/w of the demineralized water

The extract from Kalanchoe daigremontiana is an aqueous extract derived from any part of the plant, preferably the leaves and/or the juice from the leaves and/or juice from the plant or other parts of the plant biomass, such as the stalk.

The production process starts with preparation of glycerol monostearate and beeswax (the fats) which are heated up to the temperature of 30-75 degrees C. and stirred until the mixture becomes smooth.

The demineralized water is heated up to 30-75 degrees C. The fats are then added to the heated demineralized water stirred and homogenised until the mixture is smooth.

The mixture of fats and water is cooled to a temperature of 20-30 degrees C.

The extract of Kalanchoe daigremontiana is added to the mixture, stirred and homogenised to result in a smooth cream.

### Example 2

1-30% w/w of the extract or juice from Kalanchoe daigremontiana

4-20% w/w of the glycerol monostearate

4-28% w/w of the beeswax

35-75% w/w of the demineralized water

0.5-1% w/w of phenoxyethanol

The cream production process is completed as per Example 1 with the exception that in the final phase, after the extract of Kalanchoe daigremontiana is added and stirred, a preservative (phenoxyethanol) is added. The resulting mixture is then stirred and homogenised.

### Example 3

1-30% w/w of the extract or juice from Kalanchoe daigremontiana

4-20% w/w of the glycerol monostearate

4-40% w/w of the coconut oil

35-75% w/w of the demineralized water

The cream production process is as provided in Example 1 but using the alternative combination of fats as specified above.

### Example 4

1-30% w/w of the extract or juice from Kalanchoe daigremontiana

4-20% w/w of the glycerol monostearate

4-40% w/w of the coconut oil

35-75% w/w of the demineralized water

0.5-1% w/w of phenoxyethanol

The cream production process is as provided in Example 1 but using the alternative combination of fats above and in the final phase after the extract of Kalanchoe daigremontiana is added and stirred, a preservative (phenoxyethanol) is added. The resulting mixture is then stirred and homogenised.

### Example 5

1-30% w/w of the extract or juice from Kalanchoe daigremontiana

4-20% w/w of the glycerol monostearate

2-25% w/w of the cetyl alcohol

35-75% w/w of the demineralized water

The cream production process is as provided in Example 3.

### Example 6

1-30% w/w of the extract or juice from Kalanchoe daigremontiana

4-20% w/w of the glycerol monostearate

2-25% w/w of the cetyl alcohol

35-75% w/w of the demineralized water

0.5-1% w/w of phenoxyethanol

The cream production process is completed as Example 4.

### Example 7

1-30% w/w of the extract or juice from Kalanchoe daigremontiana

4-20% w/w of the glycerol monostearate

4-40% w/w of the coconut oil

2-10% w/w of the shea butter

35-75% w/w of the demineralized water

The cream production process is as provided in Example 3.

### Example 8

1-30% w/w of the extract or juice from Kalanchoe daigremontiana

4-20% w/w of the glycerol monostearate

4-40% w/w of the coconut oil

2-10% w/w of the shea butter

35-75% w/w of the demineralized water

0.5-1% w/w of phenoxyethanol

The cream production process is completed as Example 4.

### Example 9

1-30% w/w of the extract or juice from Kalanchoe daigremontiana

4-20% w/w of the glycerol monostearate

4-40% w/w of the coconut oil

0.25-0.5% w/w of the allantoin

35-75% w/w of the demineralized water

The cream production process is as provided in Example 3, with the exception that allantoin is added to the heated the demineralised water prior to the fats being added thereto.

### Example 10

1-30% w/w of the extract or juice from Kalanchoe daigremontiana

4-20% w/w of the glycerol monostearate

4-40% w/w of the coconut oil

0.25-0.5% w/w of the allantoin

35-75% w/w of the demineralized water

0.5-1% w/w of phenoxyethanol

The cream production process is as provided in Example 4, with the exception that the allantoin is added to the heated the demineralised water prior to the fats being added thereto.

### Example 11

1-30% w/w of the extract or juice from Kalanchoe daigremontiana

4-20% w/w of the glycerol monostearate

4-40% w/w of the coconut oil

2-10% w/w of the shea butter

0.25-0.5% w/w of the allantoin

35-75% w/w of the demineralized water

The cream production process is as provided in Example 9.

### Example 12

1-30% w/w of the extract or juice from Kalanchoe daigremontiana

4-20% w/w of the glycerol monostearate

4-30% w/w of the coconut oil

2-10% w/w of the shea butter

0.25-0.5% w/w of the allantoin

35-75% w/w of the demineralized water

0.5-1% w/w of phenoxyethanol

The cream production process is as provided in Example 10.

### Example 13

1-30% w/w of the extract or juice from Kalanchoe daigremontiana

4-20% w/w of the glycerol monostearate

4-40% w/w of the Cannabis sativa (Hemp) Seed Oil

2-10% w/w of the shea butter

35-75% w/w of the demineralized water

The cream production process is as provided in Example 3.

### Example 14

1-30% w/w of the extract or juice from Kalanchoe daigremontiana

4-20% w/w of the glycerol monostearate

4-40% w/w of the Cannabis sativa (Hemp) Seed Oil

2-10% w/w of the shea butter

35-75% w/w of the demineralized water

0.5-1% w/w of phenoxyethanol

The cream production process is as provided in Example 4.

### Example 15

1-30% w/w of the extract or juice from Kalanchoe daigremontiana

4-20% w/w of the glycerol monostearate

1-5% w/w of the cetyl alcohol

4-40% w/w of the coconut oil

35-75% w/w of the demineralized water

The cream production process is as provided in Example 3.

### Example 16

1-30% w/w of the extract or juice from Kalanchoe daigremontiana

4-20% w/w of the glycerol monostearate

1-5% w/w of the cetyl alcohol

4-40% w/w of the coconut oil

35-75% w/w of the demineralized water

0.5-1% w/w of phenoxyethanol

The cream production process is as provided in Example 4.

### Example 17

1-30% w/w of the extract or juice from Kalanchoe daigremontiana

4-20% w/w of the glycerol monostearate

1-5% w/w of the cetyl alcohol

4-40% w/w of the shea butter

35-75% w/w of the demineralized water

The cream production process is as provided in Example 3.

### Example 18

1-30% w/w of the extract or juice from Kalanchoe daigremontiana

4-20% w/w of the glycerol monostearate

1-5% w/w of the cetyl alcohol

4-40% w/w of the shea butter

35-75% w/w of the demineralized water

0.5-1% w/w of phenoxyethanol

The cream production process is as provided in Example 4.

### Example 19

1-30% w/w of the extract or juice from Kalanchoe daigremontiana

4-40% w/w of the Cannabis sativa (Hemp) Seed Oil

2-30% w/w of the coconut oil

35-75% w/w of the demineralized water

4-20% w/w of the glycerol monostearate

1-5% w/w of the cetyl alcohol

The cream production process is as provided in Example 3.

### Example 20

1-30% w/w of the extract or juice from Kalanchoe daigremontiana

4-40% w/w of the Cannabis sativa (Hemp) Seed Oil

2-10% w/w of the coconut oil

35-75% w/w of the demineralized water

4-20% w/w of the glycerol monostearate

1-5% w/w of the cetyl alcohol

0.5-1% w/w of phenoxyethanol

The cream production process is as provided in Example 4.

Results

Both cosmetic and clinical trials of a cream formulation comprising an extract or juice of Kalanchoe daigremontiana were conducted.

The cream that was tested comprised the formulation of within the ranges specified in Example 4.

In particular the test cream comprises:

34.4 g coconut oil (unrefined), 20 g glycerol monostearate, 35 g demineralized water, 9.6 g extract from Kalanchoe daigremontiana, 1 g phenoxyethanol.

Patient Compliance

The cream has the form of homogenous emulsion with properly selected consistency, which, in the opinion of the trial participants, enabled them to apply the cream evenly on body parts. The cream consistency and skin application ease was rated very well by the trial participants, they were able to spread it smoothly all over the skin. The participants were of the opinion that the cream is absorbed into the skin evenly and quickly (within 1 to 3 minutes) leaving a perceptible film and did not cause the feeling of stickiness or heaviness where applied.

Cosmetic Hydration

The cream significantly improves the skin hydration and does not provoke skin pulling sensation, but softens and smoothes the skin, at the same time reducing the skin roughness, itching, skin irritation, redness, rosacea and squama. The skin became supple and more pleasant to the touch after the one application of the cream.

Clinical Results and Photographic Evidence

The clinical results achieved in the following 7 symptomatic patients are provided in detail below. Further, photographic documentation, provided in the FIGS. 1 to 7, illustrates the observed effect of applying the cream with Kalanchoe daigremontiana before and after the clinical trials in each of these patients:

FIG. 1

Patient: male aged 40

Condition/symptoms: psoriasis on hands for 20 years—large patch-like psoriatic plaques, clearly separated from the healthy skin, covered with squama

(Previous treatment hospitalization and steroid treatment with some improvement to lesions)

Results: 6 weeks (twice daily)—98% clearance the psoriatic plaque receded and the exfoliation was significantly reduced 9 months following treatment.

FIG. 2

Patient: female aged 55

Condition/symptoms: psoriasis for 40 years on legs—extensive, red and dark brown psoriatic papules with big amount of stratified squama on the surface.

(Previous treatment in hospital with steroids resulted in no improvement)

Results: 12 weeks (twice daily)—95% clearance with clear reduction of the psoriatic plaques and elimination of squama 6 months following treatment.

FIG. 3

Patient: male, aged 45

Condition: psoriasis for 27 years—plaques of various sizes and shapes, with sharp, irregular edges

(Previous hospital treatment with steroids, anthralin without any long-term improvement)

Results: 10 days of daily application—visible mitigation of the psoriatic plaque

FIG. 4

Patient: female, aged 48

Condition: allergic contact dermatitis on fingers, duration of illness: 3 years

(Previous treatment with steroids resulted in no improvement)

Results: 12 days of 2 to 4 daily treatments—95% reduction in visible allergic contact dermatitis

FIGS. 5 a/b

Patient: female, aged 26

Condition/symptoms: atopic dermatitis duration of illness: 22 years on hands, neck, shoulders

(previous treatment with antihistamines, creams and ointments with no improvement)

Results: 5 days (twice daily application)—neck and shoulders improved, 8 weeks (twice daily application)—95% of atopic dermatitis cleared on hands and 100% cleared on neck and shoulders (5b—pictures on left show after)

FIG. 6

Patient: female, aged 26

Condition: psoriasis on back, duration of illness: 20 years

(Previous treatment in hospital with steroids resulted in no improvement)

Results: 9 weeks (twice daily)—100% clearance

FIG. 7

Patient: female, aged 34

Condition/symptoms: psoriasis on the head in regions of ears, duration of illness: 28 years

(Previous treatment in hospital with steroids resulted in no improvement)

Results: 5 weeks (twice daily)—85% clearance

The following general conclusions regarding patient compliance and clinical effectiveness are made by the inventors—in particular, as it concerns the effectiveness and tolerance of the cream on the face and body:
the cream soothes itching sensation after the first application (trial participants reported that itching symptoms reduced by 80% after the first application);
the cream significantly reduces or eliminates the lesions, treats irritations and inflammations (95% of the trial participants rated the cream efficacy as higher than the efficacy of other prescription-free products available on the market and clinical observations during trial and by 2 weeks of application there was reduced redness, psoriatic plaques, atopic or allergic eczema and/or the appearance of psoriatic laminae. Further, 70% of lesions presenting in patients, examined after 8-14 weeks of using the cream, found to be were clear, the remaining lesions had become paler and psoriatic laminae, atopic eczema or allergic eczema had disappeared);
the study suggests the cream reduces keratosis, eliminates squama from the surface of psoriasis lesions as early as 1 week of daily application;
the cream reduces the symptoms of skin irritation resulting from the use of detergents, chlorinated water (patients reported a feeling of relief and the feeling of soothing)
if applied regularly, it may strengthen the natural skin defence barrier; and
no adverse reactions were seen—no irritation; reddening, swelling or burning during/after application was observed or experienced by patients in the trial.

In summary, the composition of the invention therefore effectively treats dermal conditions and in particular treats the irritations such as itching, symptoms of roughness, exfoliation and the pulling and burning sensation associated with those dermal conditions. The composition is effective in speeding up regeneration and recovery of the natural protective layer of the skin.

Various further aspects and embodiments of the present invention will be apparent to those skilled in the art in view of the present disclosure. While the invention has been described herein in reference to specific aspects, features and illustrative embodiments of the invention, it will be appreciated that the utility of the invention is not thus limited, but rather extends to and encompasses variations, modifications and alternative embodiments, as will be understood by those of ordinary skill in the field.

1. A composition for topical administration comprising a therapeutically effective amount of Kalanchoe daigremontiana extract or juice.

2. The composition for topical administration of claim 1 further comprising at least one excipient for use in the treatment or prevention of a dermal condition, wherein the dermal condition is selected from the group consisting of atopic dermatitis, allergic contact dermatitis, and psoriasis.

3. (canceled)

4. A method of skin care or treating a dermal condition characterised by a combination of at least two symptoms selected from increased itching, skin redness, skin roughness, keratosis and squama, the method comprising applying the composition for topical administration of claim 2 to an affected area.

5. The composition of claim 1, wherein the composition is formulated as a powder, paste, cream, foam, gel, lotion, ointment or shampoo.

6. The composition of claim 1 wherein the composition comprises 1-30% w/w of Kalanchoe daigremontiana extract or juice.

7. The composition of claim 1 wherein the composition comprises demineralized water.

8. The composition of claim 1 wherein the composition comprises glycerol monostearate.

9. The composition of claim 2 wherein the excipient or excipients includes a mixture of fats comprising glycerol monostearate and one or more of beeswax, coconut oil, cetyl alcohol, shea butter and Cannabis sativa (Hemp) Seed Oil.

10. The composition of claim 9, wherein the composition comprises 4-28% w/w of beeswax and/or 4-40% w/w of coconut oil and/or 2-25% w/w of cetyl alcohol and/or 2-10% w/w of shea butter.

11. The composition of claim 1, wherein the composition further comprises allantoin.

12. The composition of claim 1 wherein the composition further comprises a preservative.

13. A method for making a topical composition comprising the steps of:
heating a solution of demineralized water, preferably at 30 to 75 degrees C.;
heating fat(s) comprising glycerol monostearate and stirring until smooth;
combining the fat(s) and the solution, heating the combined fat and solution, and stirring the combined fat and solution until a smooth and homogenous product is obtained;
cooling the stirred, combined fat and solution; and
adding a therapeutically effective amount of Kalanchoe daigremontiana extract or juice to the resulting mixture.

14. The method of claim 13, wherein 1-30% w/w of Kalanchoe daigremontiana extract or juice is added to the resulting mixture.

15. The method of claim 13, wherein the solution comprises 35-75% w/w demineralized water.

16. The method of claim 13, wherein the fat(s) comprises 4-20% w/w glycerol monostearate.

17. The method of claim 13 wherein the fat comprises 6-65% w/w glycerol monostearate and one or more of beeswax, coconut oil, cetyl alcohol, shea butter and Cannabis sativa (Hemp) Seed Oil.

18. The method of claim 13 wherein the fat comprises 4-28% w/w of beeswax and/or 4-40% w/w of coconut oil and/or 2-25% w/w of cetyl alcohol and/or 2-10% w/w of shea butter.

19. The method of claim 13, wherein the demineralized water is heated up to the temperature of 30-75 degrees C. and/or the mixture of fats is heated up to 30-75 degrees C.

20. The method of claim 13, wherein allantoin is added to the heated solution.

21. The method of claim 13, the method further comprising the step of adding a preservative to the resulting mixture.

22. A method of producing a topical dermal composition comprising:
combining: a) demineralized water; b) fat(s) comprising glycerol monostearate; and c) Kalanchoe daigremontiana extract or juice; and stirring to obtain a smooth homogenous composition.

23. A method of making a transdermal patch comprising: making a topical composition in accordance with the method of claim 13 and applying the composition into or on to a patch suitable for delivering the composition to the skin.

24. A transdermal patch comprising the composition of claim 1.
